(19) United States
(12) Patent Application Publication
(10) Pub. No.: US 2015/0191754 A1
(43) Pub. Date: Jul. 9, 2015

(54) METHOD FOR PREPARING SHORT-CHAIN FATTY ACID HAVING HIGH PROPANOIC ACID CONTENT BY CONTINUOUS FERMENTATION

(71) Applicant: Tongji University, Shanghai (JP)

(72) Inventors: Yinguang Chen, Shanghai (CN); Xiang Li, Shanghai (CN); Fansong Meng, Shanghai (CN)

(21) Appl. No.: 14/415,384

(22) PCT Filed: Jun. 25, 2013
(86) PCT No.: PCT/CN2013/077825

§ 371 Date: Jan. 16, 2015

(30) Foreign Application Priority Data
Jul. 18, 2012 (CN) 201210260363.X

(51) Int. Cl. C12P 7/52; C12P 7/40

(52) U.S. Cl. CPC C12P 7/52; C12P 7/40

(57) ABSTRACT

Disclosed is a method for preparing short-chain fatty acids having high propanoic acid content, comprising: mix sludge and kitchen wastes for pre-fermentation to produce substrates required for synthesis of propionic acid, and then add propionibacterium to conduct anaerobic continuous fermentation, content of propionic acid in the final fermentation broth is as high as 71.2% of the total acid.

### TECHNICAL FIELD

The present invention relates to the field of environmental protection, which relates to a method for preparing short-chain fatty acids.

### BACKGROUND ART

Previous studies show that properly increasing the content of PHV (polyhydroxyvalerate) in PHA (polyhydroxyalkanoate) can improve the plasticity of plastic, wherein, the synthesis of PHV (Biotechnol. Bioeng., 2004, 85,569-579) can be promoted by elevating propionic acid content in the raw materials for fermentation process. Meanwhile, the phosphorus removal technology adopted by wastewater treatment plant in cities is nowadays one of the key technologies for solving the problem of eutrophication. It has been reported that when the carbon molar ratio of propanoic acid/acetic acid in influent water increases from 0.16 to 2.06, the phosphorus removal efficiency increases from 68.1% to 93.5% accordingly (Water Research, 2004, 38 (1):27-36). Given that COD of influent water in sewage treatment plant in China's urban areas is relatively low, wherein the content of short-chain fatty acids(SCFAs, including acetic acid and propionic acid) is even lower, hence, to enhance the content of propionic acid in the sewage (as compared with acetic acid) is more conducive to improve the phosphorus removal effect.

It has been reported by researches that the fermentation of the sludge generated from wastewater treatment plant can produce fermentation broth rich in short-chain fatty acids (Environmental Science and Technology, 2006, 40: 2025-2029) and can elevate the content of propionic acid among the short-chain fatty acids produced from the sludge through addition of rice or kitchen wastes (Chinese invention Patent 200810035585.5; Environmental Science and Technology, 2009, 43 (12): 4373-4380). However, the content of propionic acid is at most not more than 50% and is to be further enhanced.

Currently, the main processes for the production of microbial products include continuous fermentation and batch fermentation. The principle of producing propionic acid by continuous fermentation is that by means of continuously adding culture solution into and taking out fermentation broth from fermentation tank, the microorganisms in the fermentation tank can be maintained at a certain growth stage all the time. Meanwhile, an accumulation of metabolites can be reduced, the concentration of culture solution and the content of metabolic products can remain relatively stable and the microorganisms can always stay at a stable state throughout the whole fermentation process. Compared with batch fermentation, continuous fermentation has the advantages of stable product yield and product quality, short fermentation period, high equipment utilization rate, and easy process optimization, etc.

### SUMMARY OF THE INVENTION

The present invention aims to overcome the drawbacks of the prior art and to provide a method for preparing short-chain fatty acids.

To achieve the above goals, the present invention employs the following technical solutions: Method for preparing short-chain fatty acids comprises the following steps:

(1) Mix sludge, kitchen wastes and alkali and inject into anaerobic hydrolysis fermentation tank for anaerobic fermentation;

(2) Discharge a certain volume of fermentation mixture from anaerobic fermentation reactor at a time interval, and then add fresh sludge, kitchen waste and alkali;

(3) Centrifuge discharged mixture, take out a portion of low layer solid substance after centrifugation (Remaining part is discharged outside), add water and mix in suspension oscillator, then inject together with the fresh sludge, the kitchen waste and the alkali in step (2) into the anaerobic hydrolysis fermentation tank; fetch supernatant after centrifugation to sterilize in sterilization oven, after cooling down to room temperature, adjust pH value to 6-8, and inject into anaerobic acidogenic fermentation tank;

(4) Prepare seed culture solution of propionibacterium and add into the anaerobic acidogenic fermentation tank to produce propionic acid by fermentation;

(5) Discharge a part volume of fermentation broth from the anaerobic acidogenic fermentation tank at a time interval, obtain propionic acid-rich fermentation supernatant after centrifugation, meanwhile, supplement sterilized supernatant after centrifugation and the seed culture solution of propionibacterium to the anaerobic acidogenic fermentation tank.

The alkali is calcium hydroxide (Ca(OH)₂).

The sludge in step (1) is primary sludge from wastewater treatment plant in cities or excess sludge from thickening tank, wherein, the content of total suspended solids (TSS) is 15-25 g/L, the content of volatile suspended solids/total suspended solids (VS/TS) is greater than or equal to 0.65, and the moisture content is greater than 98.0%.

The kitchen wastes in step (1) are the wastes that are grinded through pulverizer and screened from 10-mesh sieve after garbage like chopsticks, bone splinters, plastic, paper and debris are removed. Moisture content of the kitchen wastes is 65% -90%, TCOD is 75-150 g/L.

The sludge and the kitchen wastes in step (1) are mixed together according to dry weight mass ratio of 0.3:1 to 0.08:1, preferably 0.18:1; after mixing and being diluted with water, TCOD is 25-45 g/L.

Temperature of anaerobic fermentation in step (1) is 10-65° C., preferably 25° C.

The additive amount of the alkali in step (1) is equal to 0.5 -1.5% of total dry weight of mixture of the sludge and the kitchen wastes, preferably 1%.

The time interval in step (2) is 20-28 h, preferably 24 h.

Volume of discharged fermentation mixture in step (2) is ½ to ⅕ of the total volume of mixture added to reactor, preferably ⅓.

Additive volume of mixture of the fresh sludge and the kitchen wastes added in step (2) is 30-70% of the volume of discharged mixture, preferably 50%; Additive amount of the alkali added is 1-10% of dry weight of mixture of the fresh sludge and the kitchen wastes, preferably 3%.

Centrifugal speed in step (3) is 1500-3500 rpm, preferably 2500 rpm, and the centrifugation time is 2-8 min, preferably 5 min.

Sterilization temperature in step (3) is 121° C., sterilization pressure is 0.110 MPa, sterilization time is 10-30 min, preferably 20 min.

Dry weight of the low layer solid substance after centrifugation taken out in step (3) accounts for 30-70% of total dry weight of dewatered solid matters, preferably 50%.

In step (3), after addition of water, concentration of solid mixture that is injected to anaerobic hydrolysis reactor is 20-22 g/L.

Inoculation amount in step (4) is 5%-15%, preferably 10%.

Temperature for producing propionic acid by fermentation in step (4) is 25-35° C., preferably 30° C., pH value is 6-8, preferably pH=7.

The time interval in step (5) is 20-28 h, preferably 24 h.

Volume of discharged fermentation broth in step (5) is ⅓-⅕, preferably ¼ of volume of all liquids in fermentation tank.

Volume of supplemented fresh fermentation broth in step (5) is 95%-85%, preferably 90% of volume of discharged fermentation broth.

Inoculation amount of propionibacterium in step (5) is 5% -15%, preferably 10% of volume of the discharged fermentation broth.

Centrifugation speed of fermentation broth in step (5) is 500-2000 rpm, preferably 1000 rpm, and centrifugation time is 1-5 min, preferably 3 min.

Concentration of propionic acid in the fermentation supernatant in step (5) is 9.5-16.8 g COD/L.

Short chain fatty acids include acetic acid, propionic acid, isobutyric acid, n-butyric acid, isovaleric acid and n-valeric acid. The range of short-chain fatty acids is determined by specific process. In this method, the content of acetic acid in the fermentation supernatant obtained in step (5) is 2.2-4.8 g COD/L, the content of propionic acid ranges from 9.5 to 16.8 g COD/L and that of iso-butyric acid from 1.2 to 2.5 g COD/L.

Compared with the prior art, the present invention has the following advantageous and beneficial effects:

(1) The substrates required for synthesis of propionic acid are produced by facilitating pre-fermentation of sludge under the weak alkali condition and using kitchen wastes.

(2) Volatile substances in sludge and kitchen wastes decrease nearly by 30%.

(3) The content of propionic acid in the final fermentation broth of sludge is 71.2% (COD equivalence ratio).

### BRIEF DESCRIPTION OF THE DRAWINGS

FIG. 1 shows the flow chart of process for preparing short-chain fatty acids having high propanoic acid content by continuous fermentation in the embodiments of the present invention.

### DETAILED DESCRIPTIONS OF THE PREFERRED EMBODIMENTS

The inventions are further described herein in connection with certain specific embodiments and attached drawings.

The detection indicators for fermentation supernatant include the total volume of filtrate, short-chain fatty acids (SCFAs), VSS, and etc.

### Embodiment 1

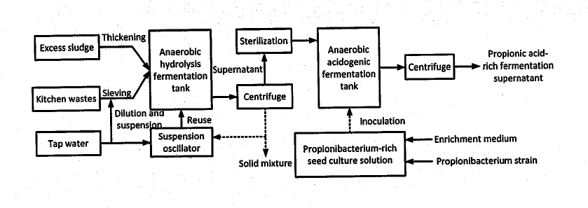

(1) As is shown in FIG. 1, fetch 6 L excess sludge from thickening tank of wastewater treatment plant in cities (TSS 20 g/L, VSS 14 g/L), 5.3 L kitchen wastes from canteens (excluding chopsticks, bone splinters, plastic, paper, debris and other wastes, being grinded through a pulverizer and screened from a 10-mesh sieve to obtain a moisture content of 85%, TCOD of 96 g/L), respectively, with the mass ratio of dry weight of sludge and kitchen wastes at 0.18:1, add 15.7 L of tap water and inject into a 30 L of anaerobic hydrolysis fermentation tank (volume of mixture in the tank is approximately 27 L), the initial TCOD is 30 gCOD/L; at the same time, add 7.8 g of Ca(OH)₂ (i.e., the mass of the Ca(OH)₂ is 1% of the dry weight of the mixture added), stir at room temperature (25° C.) for anaerobic fermentation;

(2) Discharge 9 L mixture (accounting for ⅓ of the total volume of mixture) from anaerobic hydrolysis fermentation reactor at a time interval of 24 h, and introduce 4.5 L mixture of fresh sludge and kitchen waste (the ratio of introduced fresh substances account for 50% of the volume of discharged mixture) and 3.9 g Ca(OH)₂ (3% of the dry weight of the introduced fresh mixture) into anaerobic fermentation reactor;

(3) Centrifuge discharged mixture at 2500 rpm for 5 min to obtain about 8.5 L of fermentation supernatant and 0.5 L dewatered sludge cake. 0.25 L of dewatered sludge cake (accounting for 50% of total dewatered sludge cake) and 4.25 L of tap water are injected into the suspension oscillator for oscillation; after fully mixing (solid concentration of 20.97 g/L), add into anaerobic hydrolysis fermentation tank; 8.5 L of supernatant is injected to the sterilization oven for sterilization for 20 min at the temperature of 121° C. and at the pressure of 0.110 MPa; after cooling down to room temperature, the pH value is adjusted to 7 by Ca(OH)₂ emulsion, and pour into 40 L of anaerobic acidogenic fermentation tank;

(4) Prepare 3.3 L seed culture solution of propionibacterium (Enrichment for 4 days is required according to attached table 1. Within the 4 days, continue to accumulate the dewatered supernatant in the anaerobic acidogenic fermentation tank to 33 L), inoculate 10% to the anaerobic acidogenic fermentation tank, and maintain pH=7 with Ca(OH)₂ emulsion for anaerobic fermentation under the condition of 30° C.;

(5) Discharge 9 L (¼ of total volume) fermentation broth from the anaerobic acidogenic fermentation tank at a time interval of 24 h, and add 8 L (the inoculation amount of propionibacterium is 90% of volume of discharged fermentation broth) of sterilized and dewatered supernatant and 1 L of well prepared seed solution of propionibacterium (the inoculation amount of propionibacterium is 10% of volume of the discharged fermentation broth) into the anaerobic acidogenic fermentation tank. The discharged fermentation broth is centrifuged for 5 min at 1000 rpm to get 8.6 L supernatant of propionic acid-rich fermentation broth.

After analysis, volatile organic substances of the mixture of sludge and kitchen wastes can be reduced by 27.8%, and propionic acid of the final fermentation supernatant is 16.76 g COD/L, accounting for 71.2% of the total acids.

Embodiment 2

(1) Fetch 9 L excess sludge from thickening tank of wastewater treatment plant in cities (TSS 20 g/L, VSS 14 g/L), 4.8 L kitchen wastes from canteens (excluding chopsticks, bone splinters, plastic, paper, debris and other wastes, being grinded through a pulverizer and screened from a 10-mesh sieve to obtain a moisture content of 65%, TCOD of 150 g/L), respectively add 13.2 L of tap water with the mass ratio of dry weight of sludge and kitchen wastes at 0.3:1, and inject into a 30 L of anaerobic hydrolysis fermentation tank (volume of mixture in the tank is approximately 27 L), the initial TCOD is 30 gCOD/L; at the same time, add 11.8 g of Ca(OH)₂ (i.e., the mass of the Ca(OH)₂ is 1.5% of the dry weight of the mixture added), stir at room temperature (25° C.) for anaerobic fermentation;

(2) Discharge 9 L mixture (accounting for ⅓ of the total volume of mixture) from anaerobic hydrolysis fermentation reactor at a time interval of 28 h, and introduce 6.3 L mixture of fresh sludge and kitchen waste (the ratio of introduced fresh substances account for 50%) and 18.4 g Ca(OH)₂ (10% of the dry weight of the introduced fresh mixture) into anaerobic fermentation reactor;

(3) Centrifuge discharged mixture at 1500 rpm for 2 min to obtain about 8.5 L of fermentation supernatant and 0.5 L dewatered sludge cake. 0.15 L of dewatered sludge cake (accounting for 30% of total dewatered sludge cake) and 2.55 L of tap water are injected into the suspension oscillator for oscillation; after fully mixing (solid concentration of 21.42 g/L), add into anaerobic hydrolysis fermentation tank; 8.5 L of supernatant is injected to the sterilization oven for sterilization for 10 min at the temperature of 121° C. and at the pressure of 0.110 MPa; after cooling down to room temperature, the pH value is adjusted to 7 by Ca(OH)₂ emulsion, and pour into 40 L of anaerobic acidogenic fermentation tank;

(4) Prepare 1.6 L seed culture solution of propionibacterium (Enrichment for 4 days is required. Within the 4 days, accumulate the dewatered supernatant to 32 L), inoculate 5% to the anaerobic acidogenic fermentation tank, and maintain pH=8 with Ca(OH)₂ emulsion for anaerobic fermentation under the condition of 35° C.;

(5) Discharge 8.4 L (¼ of total volume) fermentation mixture from the anaerobic acidogenic fermentation tank at a time interval of 28 h, and add 8 L (accounting for 95% of discharged volume) of sterilized and dewatered supernatant and 0.4 L (accounting for 5% of discharged volume) of well prepared seed solution of propionibacterium into the anaerobic acidogenic fermentation tank. The discharged fermentation mixture is centrifuged for 1 min at 500 rpm to get 8 L supernatant of propionic acid-rich fermentation broth.

After analysis, volatile organic substances of the mixture of sludge and kitchen wastes can be reduced by 26.5%, and propionic acid of the final fermentation supernatant is 9.54 g COD/L (accounting for 62.5% of the total acids).

### Embodiment 3

(1) Fetch 2.9 L excess sludge from thickening tank of wastewater treatment plant in cities (TSS 20 g/L, VSS 14 g/L), 5.8 L kitchen wastes from canteens (excluding chopsticks, bone splinters, plastic, paper, debris and other wastes, being grinded through a pulverizer and screened from a 10-mesh sieve to obtain a moisture content of 90%, TCOD of 75 g/L), respectively add 18.3 L of tap water with the mass ratio of dry weight of sludge and kitchen wastes at 0.08:1, and inject into a 30 L of anaerobic hydrolysis fermentation tank, the initial TCOD is 30 gCOD/L; at the same time, add 3.9 g of Ca(OH)₂ (i.e., the mass of the Ca(OH)₂ is 0.5% of the dry weight of the mixture added), stir at room temperature (25° C.) for anaerobic fermentation;

(2) Discharge 9 L mixture (accounting for ⅓ of the total volume of mixture) from anaerobic hydrolysis fermentation reactor at a time interval of 20 h, and introduce 2.7 L mixture of fresh sludge and kitchen waste (the ratio of introduced fresh substances account for 30%) and 0.78 g Ca(OH)₂ (mass of Ca(OH)₂ accounts for 1% of the dry weight of the introduced fresh mixture) into anaerobic fermentation reactor;

(3) Centrifuge discharged fermentation mixture at 3500 rpm for 8 min to obtain about 8.5 L of fermentation supernatant and 0.5 L dewatered sludge cake. 0.35 L of dewatered sludge cake (accounting for 70% of total dewatered sludge cake) and 5.95 L of tap water are injected into the suspension oscillator for oscillation; after fully mixing (solid concentration of 21.87 g/L), add into anaerobic hydrolysis fermentation tank; 8.5 L of dewatered supernatant is injected to the sterilization oven for sterilization for 30 min at the temperature of 121° C. and at the pressure of 0.110 MPa; after cooling down to room temperature, the pH value is adjusted to 7 by Ca(OH)₂ emulsion, and pour into 40 L of anaerobic acidogenic fermentation tank;

(4) Prepare 4 L seed culture solution of propionibacterium (Enrichment for 4 days is required. Within the 4 days, continue to accumulate the dewatered supernatant in the anaerobic acidogenic fermentation tank to 26 L), inoculate 15% to the anaerobic acidogenic fermentation tank, and maintain pH=6 with Ca(OH)₂ emulsion for anaerobic fermentation under the condition of 25° C.;

(5) Discharge 10 L (⅓ of total volume) fermentation broth from the anaerobic acidogenic fermentation tank at a time interval of 20 h, and add 8.5 L (accounting for 85% of discharged volume) of sterilized and dewatered supernatant and 1.5 L (accounting for 15% of discharged volume) of well prepared seed solution of propionibacterium into the anaerobic acidogenic fermentation tank. The discharged fermentation broth is centrifuged for 5 min at 1000 rpm to get 9.5 L supernatant of propionic acid-rich fermentation broth.

After analysis, volatile organic substances of the mixture of sludge and kitchen wastes can be reduced by 24.5%, and propionic acid of the final fermentation supernatant is 15.78 g COD/L(accounting for 64.2% of the total acids).

### Embodiment 4

(1) Fetch 6 L excess sludge from thickening tank of wastewater treatment plant in cities (TSS 20 g/L, VSS 14 g/L), 5.3 L kitchen wastes from canteens (excluding chopsticks, bone splinters, plastic, paper, debris and other wastes, being grinded through a pulverizer and screened from a 10-mesh sieve to obtain a moisture content of 85%, TCOD of 96 g/L), respectively add 15.7 L of tap water with the mass ratio of dry weight of sludge and kitchen wastes at 0.18:1, and inject into a 30 L of anaerobic hydrolysis fermentation tank, the initial TCOD is 30 gCOD/L; at the same time, add 7.8 g of Ca(OH)₂ (i.e., the mass of the Ca(OH)₂ is 1% of the dry weight of the mixture added), stir at temperature of 10° C. for anaerobic fermentation;

(2) Discharge 5.4 L mixture (accounting for ⅕ of the total volume of mixture) from anaerobic hydrolysis fermentation reactor at a time interval of 24 h, and introduce 2.7 L mixture of fresh sludge and kitchen waste (the ratio of introduced fresh substances account for 50%) and 2.4 g Ca(OH)₂ (mass of Ca(OH)₂ accounts for 3% of the dry weight of the introduced fresh mixture) into anaerobic fermentation reactor;

(3) Centrifuge discharged fermentation mixture at 2500 rpm for 5 min to obtain about 5 L of fermentation supernatant and 0.4 L dewatered sludge cake. 0.2 L of dewatered sludge cake (accounting for 50% of total dewatered sludge cake) and 2.5 L of tap water are injected into the suspension oscillator for oscillation; after fully mixing (solid concentration of 21.71 g/L), add into anaerobic hydrolysis fermentation tank; supernatant is injected to the sterilization oven for sterilization for 20 min at the temperature of 121° C. and at the pressure of 0.110 MPa; after cooling down to room temperature, the pH value is adjusted to 7 by Ca(OH)₂ emulsion, and pour into 40 L of anaerobic acidogenic fermentation tank;

(4) Prepare 2.1 L seed culture solution of propionibacterium (Enrichment for 4 days is required. Within the 4 days, continue to accumulate the dewatered supernatant in the anaerobic acidogenic fermentation tank to 21 L), inoculate 10% to the anaerobic acidogenic fermentation tank, and maintain pH=7 with Ca(OH)₂ emulsion for anaerobic fermentation under the condition of 30° C.;

(5) Discharge 4.6 L (⅕ of total volume) fermentation mixture from the anaerobic acidogenic fermentation tank at a time interval of 24 h, and add 4.1 L (accounting for 90% of discharged volume) of sterilized and dewatered supernatant and 0.5 L (accounting for 10% of discharged volume) of well prepared seed solution of propionibacterium into the anaerobic acidogenic fermentation tank. The discharged mixture is centrifuged for 5 min at 1000 rpm to get 4.4 L supernatant of propionic acid-rich fermentation broth.

After analysis, volatile organic substances of the mixture of sludge and kitchen wastes can be reduced by 25.3%, and propionic acid of the final fermentation supernatant is 13.14 g COD/L(accounting for 68.3% of the total acids).

### Embodiment 5

(1) Fetch 6 L excess sludge from thickening tank of wastewater treatment plant in cities (TSS 20 g/L, VSS 14 g/L), 5.3 L kitchen wastes from canteens (excluding chopsticks, bone splinters, plastic, paper, debris and other wastes, being grinded through a pulverizer and screened from a 10-mesh sieve to obtain a moisture content of 85%, TCOD of 96 g/L), respectively add 15.7 L of tap water with the mass ratio of dry weight of sludge and kitchen wastes at 0.18:1, and inject into a 30 L of anaerobic hydrolysis fermentation tank, the initial TCOD is 30 gCOD/L; at the same time, add 7.8 g of Ca(OH)₂ (i.e., the mass of the Ca(OH)₂ is 1% of the dry weight of the mixture added), stir at temperature of 65° C. for anaerobic fermentation;

(2) Discharge 13.5 L mixture (accounting for ½ of the total volume of mixture) from anaerobic hydrolysis fermentation reactor at a time interval of 24 h, and introduce 4.05 L mixture of fresh sludge and kitchen waste (the ratio of introduced fresh substances account for 30%) and 3.53 g Ca(OH)₂ (mass of Ca(OH)₂ accounts for 3% of the dry weight of the introduced fresh mixture) into anaerobic fermentation reactor;

(3) Centrifuge discharged fermentation mixture at 2500 rpm for 5 min to obtain about 12.2 L of fermentation supernatant and 1.3L dewatered sludge cake. 0.9 L of dewatered sludge cake (accounting for 70% of total dewatered sludge cake) and 8.55 L of tap water are injected into the suspension oscillator for oscillation; after fully mixing (solid concentration of 21.85 g/L), add into anaerobic hydrolysis fermentation tank; supernatant is injected to the sterilization oven for sterilization for 20 min at the temperature of 121° C. and at the pressure of 0.110 MPa; after cooling down to room temperature, the pH value is adjusted to 7 by Ca(OH)₂ emulsion, and pour into 40 L of anaerobic acidogenic fermentation tank;

(4) Prepare 3.3 L seed culture solution of propionibacterium (Enrichment for 4 days is required. Within the 4 days, accumulate the dewatered supernatant to 33 L), inoculate 10% to the anaerobic acidogenic fermentation tank, and maintain pH=7 with Ca(OH)₂ emulsion for anaerobic fermentation under the condition of 30° C.;

(5) Discharge 12.1 L (⅓ of total volume) mixture from the anaerobic acidogenic fermentation tank at a time interval of 24 h, and add 10.9 L (accounting for 90% of discharged volume) of sterilized and dewatered supernatant and 1.2 L (accounting for 10% of discharged volume) of well prepared seed solution of propionibacterium into the anaerobic acidogenic fermentation tank. The discharged mixture is centrifuged for 5 min at 1000 rpm to get 11.5 L supernatant of propionic acid-rich fermentation broth.

After analysis, volatile organic substances of the mixture of sludge and kitchen wastes can be reduced by 24.8%, and propionic acid of the final fermentation supernatant is 12.53 g COD/L(accounting for 68.3% of the total acids).

According to the test data given in the above embodiments 1-5, the present invention has the following advantages as compared with prior arts:

1. The method for producing propionic acid by combination of sludge and kitchen wastes has not been reported yet.

2. As compared with some other methods for producing propionic acid by pure bacteria, this new method recycles the wastes as resources, while the substrates adopted by present propionic acid-producing technologies are pure chemical agents with higher costs.

3. Compared with the existing methods for production of acid by sludge fermentation, the acid yield through this method is greatly enhanced, up to 16.7 g COD/L propionic acid can be obtained and the content of propionic acid is up to 71%; whereas, the content of propionic acid obtained by traditional methods for production of acid by anaerobic alkali fermentationis only 1-3 g COD/Land the highest content only 20%.

4. Compared with the traditional methods of fermentation of sludge and kitchen wastes, the acid yield by this method is greatly enhanced, up to 16.7 g COD/L propionic acid can be obtained and the content of propionic acid is up to 71%; whereas, the content of propionic acid obtained by traditional methods for production of acid by anaerobic weak alkali (pH=8) fermentation of sludge and kitchen wastes is only 5-8 g COD/L and the highest content only 48%.

Table 1 shows the enrichment medium for propionibacterium in the embodiments of the present invention

TABLE 1

| Component | Concentration (g/L) | Substitute |
|---|---|---|
| Yeast extract | 10 | Corn syrup |
| Casein enzymatic | 10 | Pancreatic digest of casein |
| hydrolysate | | |
| K2HPO4•H2O | 2.5 | |
| KH2PO4 | 1.5 | |
| Glucose | 20 | Sodium lactate |
| pH | 6.8-7.2 | Adjusted with KOH/HCl |

The above descriptions of embodiments are conducive for ordinary technicians of the present technical field to understand and apply the invention. It is obvious that persons skilled in the art of the present field can easily make various amendments to the above embodiments and apply the general principle illustrated in here into other embodiments without the effort of inventive work. Therefore, the present invention is not confined to embodiments herein. Any improvements and modifications conducted by persons skilled in the art of the present field according to the instructions of the present invention and without going beyond the scope of the present invention shall be included in the extent of protection of the present invention.

What is claimed is:

1. A method for preparing short-chain fatty acids having high propanoic acid content by continuous fermentation, comprising the following steps:
(1) Mix sludge, kitchen wastes and alkali for anaerobic fermentation;
(2) Discharge a certain volume of fermentation mixture from anaerobic fermentation reactor at a time interval, and then add fresh sludge, kitchen waste and alkali;
(3) Centrifuge discharged mixture, take out a portion of low layer solid substance after centrifugation, add water and mix in suspension oscillator, then inject together with the fresh sludge, the kitchen waste and the alkali in step (2) into anaerobic hydrolysis fermentation tank; fetch supernatant after centrifugation to sterilize in sterilization oven, after cooling down to room temperature, adjust pH value to 6-8, and inject into anaerobic acidogenic fermentation tank;
(4) Prepare seed culture solution of propionibacterium, and add into the anaerobic acidogenic fermentation tank to produce propionic acid by fermentation;
(5) Discharge a part volume of fermentation broth from the anaerobic acidogenic fermentation tank at a time interval, obtain propionic acid-rich fermentation supernatant after centrifugation, meanwhile, supplement sterilized supernatant after centrifugation and the seed culture solution of propionibacterium to the anaerobic acidogenic fermentation tank.

2. The method according to claim 1, wherein the alkali is calcium hydroxide (Ca(OH)₂);
or the sludge in step (1) is primary sludge from wastewater treatment plant in cities or excess sludge from thickening tank, wherein, the content of total suspended solids (TSS) is 15-25 g/L, the content of volatile suspended solids/total suspended solids (VS/TS) is greater than or equal to 0.65, and the moisture content is greater than 98.0%;
or the kitchen wastes in step (1) are the wastes that are grinded through pulverizer and screened from 10-mesh sieve after chopsticks, bone splinters, plastic, paper and debris are removed, moisture content of the kitchen wastes is 65% -90%, TCOD is 75-150 g/L.

3. The method according to claim 1, wherein the sludge and the kitchen wastes in step (1) are mixed together according to dry weight mass ratio of 0.3:1 to 0.08:1, preferably 0.18:1; after mixing and being diluted with water, TCOD is 25-45 g/L.

4. The method according to claim 1, wherein temperature of anaerobic fermentation in step (1) is 10-65° C., preferably 25° C.;
or the additive amount of the alkali in step (1) is equal to 0.5 -1.5% of total dry weight of mixture of the sludge and the kitchen wastes, preferably 1%.

5. The method according to claim 1, wherein the time interval in step (2) is 20-28 h, preferably 24 h;
or volume of discharged fermentation mixture in step (2) is ½-⅗ of the total volume of mixture added to reactor, preferably ⅓.
or additive volume of mixture of the fresh sludge and the kitchen wastes added in step (2) is 30-70% of the volume of discharged mixture, preferably 50%; Additive amount of the alkali added is 1-10% of dry weight of mixture of the fresh sludge and the kitchen wastes, preferably 3%.

6. The method according to claim 1, wherein centrifugal speed in step (3) is 1500-3500 rpm, preferably 2500 rpm, and the centrifugation time is 2-8 min, preferably 5 min;
or sterilization temperature in step (3) is 121° C., sterilization pressure is 0.110 MPa, sterilization time is 10-30 min, preferably 20 min.

7. The method according to claim 1, wherein dry weight of the low layer solid substance taken out in step (3) accounts for 30-70% of total dry weight of dewatered solid matters, preferably 50%;
or in step (3), after addition of water, concentration of solid mixture that is injected to anaerobic hydrolysis reactor is 20-22 g/L.

8. The method according to claim 1, wherein inoculation amount in step (4) is 5% -15%, preferably 10%;
or temperature for producing propionic acid by fermentation in step (4) is 25-35° C., preferably 30° C., pH value is 6-8, preferably pH=7.

9. The method according to claim 1, wherein the time interval in step (5) is 20-28 h, preferably 24 h;
or volume of discharged fermentation broth in step (5) is ⅓-⅕, preferably ¼ of volume of all liquids in fermentation tank.
or volume of supplemented fresh fermentation broth in step (5) is 95% to 85%, preferably 90% of volume of discharged fermentation broth.

10. The method according to claim 1, wherein inoculation amount of propionibacterium in step (5) is 5%-15%, preferably 10% of volume of the discharged fermentation broth;
or centrifugation speed of fermentation broth in step (5) is 500-2000 rpm, preferably 1000 rpm, and centrifugation time is 1-5 min, preferably 3 min;
or concentration of propionic acid in the fermentation supernatant in step (5) is 9.5-16.8 g COD/L.
